(19) United States
(12) Patent Application Publication
(10) Pub. No.: US 2025/0172574 A1
(43) Pub. Date: May 29, 2025

(54) REAGENT FOR DETECTING METHYL SALICYLATE, METHYL SALICYLATE SENSOR, METHOD FOR SENSING METHYL SALICYLATE USING THE SAME AND METHODS FOR DETECTING PLANT PATHOGEN INFECTIONS

(71) Applicant: NEC Corporation, Tokyo (JP)

(72) Inventor: Katsumi Maeda, Tokyo (JP)

(73) Assignee: NEC Corporation, Tokyo (JP)

(21) Appl. No.: 18/937,250

(22) Filed: Nov. 5, 2024

(30) Foreign Application Priority Data
Nov. 29, 2023 (JP) 2023-201283

(51) Int. Cl. G01N 33/74; G01N 33/533

(52) U.S. Cl. CPC G01N 33/74; G01N 33/533; G01N 2333/415

(57) ABSTRACT

The present disclosure provides a reagent for detecting methyl salicylate, which is a plant hormone released when a plant is infected with a disease, in the cultivation of plants including agricultural crops, a methyl salicylate sensor, and a method for sensing methyl salicylate, using the same, thereby providing a method for detection of pathogen infection in a plant.

A reagent for detecting methyl salicylate, comprising a terbium compound doped with a rare earth element other than terbium. A methyl salicylate sensor comprising a capture section of methyl salicylate having the reagent and a detection section detecting that methyl salicylate has been captured by the capture section.

This application is based upon and claims the benefit of priority from Japanese patent application No. 2023-201283, filed on Nov. 29, 2023, the disclosure of which is incorporated herein in its entirety by reference.

### TECHNICAL FIELD

The present disclosure relates to a reagent for detecting methyl salicylate, which is a plant hormone released when plants are infected with pathogens, a methyl salicylate sensor, a method for sensing methyl salicylate using the same, and a method for detecting pathogen infection in a plant.

### BACKGROUND ART

It is known that when a plant is infected with a pathogen, it synthesizes and releases a plant hormone, which is a signal substance, and informs surrounding plants of the pathogen infection, thereby promoting a defense mechanism in advance. By quickly recognizing a signal substance emitted by such plant, it becomes possible to detect pest damage.

As a method for detection of pest damage to a target plant, for example, Patent document 1 describes a method in which a monitor plant having a photoprotein gene is placed in the vicinity of a target plant, and the monitor plant senses volatile substances released by the target plant in response to stress and emits light.

### CITATION LIST

### Patent Literature

Patent Literature 1: International Publication No. WO2019/082942

### SUMMARY OF INVENTION

### Technical Problem

The object of the present disclosure is to provide a reagent for detecting methyl salicylate, which is a plant hormone released when a plant is infected with a disease, in the cultivation of plants including agricultural crops, a methyl salicylate sensor, and a method for sensing methyl salicylate using the same, thereby providing a method for detecting pathogen infection in a plant.

### Solution to Problem

The present inventors have made intensive studies to solve the above problems. As a result, by using a reagent comprising a terbium compound doped with a rare earth element other than terbium (hereinafter also referred to as “other rare earth element”) as a receptor for methyl salicylate, fluorescence emission derived from the complex generated upon reaction of the terbium compound with methyl salicylate is increased compared to the case where the reagent containing an undoped terbium compound is used, and the pathogen infection in a plant can be detected, and the present disclosure has been completed.

One aspect of this embodiment relates to a reagent for detecting methyl salicylate, comprising a terbium compound doped with a rare earth element other than terbium.

Further, one aspect of this embodiment relates to a methyl salicylate sensor for detecting methyl salicylate comprising:
a capture section of methyl salicylate having a reagent comprising a terbium compound doped with a rare earth element other than terbium, and
a detection section detecting that methyl salicylate has been captured by the capture section.

Moreover, one aspect of this embodiment relates to a method for sensing methyl salicylate, comprising detecting methyl salicylate using the reagent, comprising:
(i) reacting a terbium compound doped with rare earth element other than terbium with methyl salicylate to form a complex;
(ii) exposing the complex to excitation light; and
(iii) detecting fluorescence emitted by the complex.

Furthermore, one aspect of this embodiment relates to a method for detecting pathogen infection in a plant by placing the reagent in the vicinity of a plant and confirming fluorescence emission derived from a complex formed by a reaction between a terbium compound doped with a rare earth element other than terbium and methyl salicylate.

### Advantageous Effect of Invention

According to the present disclosure, the intensity of fluorescence emission derived from the complex formed by the reaction of methyl salicylate, which is a volatile plant hormone released when plants are infected with pathogens and the terbium compound, is increased by including a terbium compound doped with a rare earth element other than terbium (i.e., other rare earth element) as a receptor for methyl salicylate in a reagent for detecting methyl salicylate, compared to the case where an undoped terbium compound is contained, making it possible to detect infection of plants by pathogenic bacteria.

### BRIEF DESCRIPTION OF DRAWINGS

FIG. 1 shows the fluorescence spectrum curves obtained in the present disclosure.

FIG. 2 shows the fluorescence spectrum curves obtained in the present disclosure.

FIG. 3 shows the fluorescence spectrum curves obtained in the present disclosure.

FIG. 4 shows the fluorescence spectrum curves obtained in the present disclosure.

FIG. 5 shows the fluorescence spectrum curves obtained in the present disclosure.

FIG. 6 shows the fluorescence spectrum curves obtained in the present disclosure.

### DESCRIPTION OF EMBODIMENTS

Hereinafter, the embodiment of the present disclosure will be described with reference to the drawings. However, the embodiment described below has technically preferable limitations for carrying out the present disclosure, but the scope of the invention is not limited to the following.

### [1] Reagent for Detecting Methyl Salicylate

One embodiment of the present disclosure is a reagent for detecting methyl salicylate, comprising a terbium compound doped with a rare earth element other than terbium as a receptor for methyl salicylate. In the present disclosure, the term “reagent” is defined as a chemical substance used for the detection or quantification of substances by chemical methods, the synthesis of substances or the measurement of physical properties.

In addition, the reagent preferably comprises a non-volatile ionic liquid that effectively captures methyl salicylate in addition to the terbium compound doped with other rare earth element. When the reagent comprises an ionic liquid, the terbium compound doped with other rare earth element is dissolved in the non-volatile ionic liquid, but may be partially precipitated. The terbium compound doped with other rare earth element can function as a receptor for methyl salicylate in the same manner, whether dissolved in the non-volatile ionic liquid or precipitated.

<Receptor for Methyl Salicylate: Terbium Compound Doped with Other Rare Earth Element>

The compound represented by the following general formula (1) is used as the terbium compound doped with other rare earth element that can be used to recognize methyl salicylate in the present disclosure.

Ln_{c}TbXₐY_{b} (1)
wherein Ln represents at least one rare earth element other than terbium, X and Y each independently represent at least one selected from the group consisting of acetate ion, propionate ion, butyrate ion, pivalate ion, 2-methylbutyrate ion, trifluoroacetate ion, benzoate ion, chloride ion, and nitrate ion, c represents a number ranging from 0.015 to 0.05, and a and b each independently represent a number that satisfies 0≤a≤3.15, 0≤b≤3.15, and a+b=3+3×c.

When two or more rare earth elements other than terbium are doped, the sum of them, c, is 0.015 to 0.05. When c is in this range, the effect of increasing the intensity of fluorescence emission derived from the complex formed by the reaction with methyl salicylate is sufficiently exhibited.

Specific examples of other rare earth elements include scandium, yttrium, lanthanum, cerium, praseodymium, neodymium, promethium, samarium, europium, gadolinium, dysprosium, holmium, erbium, thulium, ytterbium, and lutetium.

Specific examples of terbium compounds doped with other rare earth element include, but are not limited to, Ce_{0.02}Tb(CH₃COO)_{0.81}(t-C₄H₉COO)_{2.25}, Eu_{0.02}Tb(CH₃COO)_{0.81}(t-C₄H₉COO)_{2.25}, Sm_{0.02}Tb(CH₃COO)_{0.81} (t-C₄H₉COO)_{2.25}, Dy_{0.02}Tb(CH₃COO)_{0.81} (t-C₄H₉COO)_{2.25}, Gd_{0.02}Tb(CH₃COO)_{0.81} (t-C₄H₉COO)_{2.25}, Yb_{0.02}Tb(CH₃COO)_{0.81} (t-C₄H₉COO)_{2.25}, Ce_{0.01}Tb(CH₃COO)_{0.78} (t-C₄H₉COO)_{2.25}, Ce_{0.015}Tb(CH₃COO)_{0.795} (t-C₄H₉COO)_{2.25}, Ce_{0.025}Tb(CH₃COO)_{0.825} (t-C₄H₉COO)_{2.25}, Ce_{0.04}Tb(CH₃COO)_{0.87} (t-C₄H₉COO)_{2.25}, Ce_{0.05}Tb(CH₃COO)_{0.9}(t-C₄H₉COO)_{2.25}, Ce_{0.06}Tb(CH₃COO)_{0.93} (t-C₄H₉COO)_{2.25}, Ce_{0.02}Tb(CH₃COO)_{3.06}. Ce_{0.04}Tb(CH₃COO)_{3.12}, Yb_{0.15}Tb(CH₃COO)_{0.795} (t-C₄H₉COO)_{2.25}, Yb_{0.025}Tb(CH₃COO)_{0.825} (t-C₄H₉COO)_{2.25}, Yb_{0.04}Tb(CH₃COO)_{0.87} (t-C₄H₉COO)_{2.25}, Ce_{0.02}Tb(NO₃)_{3.06} and Yb_{0.02}Tb(NO₃)_{3.06}.

The terbium compound doped with other rare earth element, for example, cerium-doped terbium acetate-terbium pivalate complex salt (Ce_{c}Tb(CH₃COO)ₐ(t-C₄H₉COO)_{b}) can be synthesized by dissolving terbium acetate, terbium pivalate, and any number of moles of cerium acetate relative to terbium (Tb) in methanol and reacting them under heat. In addition, by changing the ratio of the two terbium salts (terbium acetate and terbium pivalate) and the molar ratio of cerium acetate during synthesis, the ratio (a, b) of carboxylate ions (X, Y) and the doping amount of cerium (c) can be changed.

The terbium compound doped with other rare earth element reacts with methyl salicylate to form a complex, and is thereby able to selectively recognize methyl salicylate. Compared with a terbium compound not doped with other rare earth element, the terbium compound doped with other rare earth element exhibits increased fluorescence emission intensity derived from the complex formed by the reaction with methyl salicylate, making it easier to sense methyl salicylate and enabling the detection of infection of plants by pathogenic bacteria.

The resulting complex emits a complex-specific fluorescence when excited by UV light. If only the terbium compound doped with other rare earth element is used, the fluorescence emission intensity is small even when irradiated with UV light, so it is not observed. Furthermore, the terbium compound does not react with and recognize other plant hormones than methyl salicylate, such as methyl jasmonate, so it can selectively recognize methyl salicylate.

### <Non-Volatile Ionic Liquid>

In order to effectively capture methyl salicylate, the reagent of the present disclosure may comprise a non-volatile ionic liquid in addition to the erbium compound doped with other rare earth element. Non-volatile ionic liquids that can be used in the present disclosure include imidazolium salts, phosphonium salts, pyridinium salts, ammonium salts, piperidinium salts, pyrrolidinium salts and so on. Specific examples of the non-volatile ionic liquid include 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butyl-3-methylimidazolium dibutyl phosphate, tetrabutyl ammonium acetate, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide and 1-butyl-3-methylimidazolium dicyanamide, but are not limited thereto.

The ratio of the ionic liquid to the terbium compound doped with other rare earth element is preferably 2 to 40 times by weight, more preferably 3 to 30 times by weight, and particularly preferably 4 to 20 times by weight. If the weight ratio of the ionic liquid to the terbium compound doped with other rare earth element is less than 2 times, the effect of improving the detection sensitivity of methyl salicylate may not be obtained. If the weight ratio of the ionic liquid to the terbium compound doped with other rare earth element exceeds 40 times, the terbium compound doped with other rare earth element may be difficult to form a complex with methyl salicylate.

The reagent for detecting methyl salicylate of the present disclosure may comprise any other solvent within the range that does not impair the effect of the present disclosure. Examples of the solvent that can be used include, but are not limited to, dimethyl sulfoxide, methanol, ethanol, water, N,N-dimethylformamide, tetrahydrofuran, acetone, acetonitrile and 1,4-dioxane.

### [2] Methyl Salicylate Sensor

One embodiment of the present disclosure relates to a methyl salicylate sensor for detecting methyl salicylate, comprising a capture section of methyl salicylate having a reagent comprising a terbium compound doped with rare earth element other than terbium, and a detection section detecting that methyl salicylate has been captured by the capture section.

### (1) Capture Section

The capture section of the methyl salicylate sensor of the present disclosure has a reagent comprising a terbium compound doped with other rare earth element, which is a receptor that selectively captures methyl salicylate. In the capture section, the reagent is preferably comprised in a medium.

### <Medium>

The medium containing the terbium compound doped with other rare earth element of the present disclosure may be, includes, but is not limited to, for example, paper or glass fibers, resins (for example, polymethyl methacrylate, polyethylene, polypropylene, polyvinyl chloride, polystyrene, nylon resin, polyamide, polycarbonate, polyethylene terephthalate, polybutylene terephthalate, polyphenylene oxide), water soluble polymers (cellulose, agarose, starch, sodium alginate, acrylic acid-, acrylamide, polyvinyl alcohol, polyethylene oxide, polyvinylpyrrolidone).

For example, when paper is used as a medium, a terbium compound doped with other rare earth element is dissolved in a solvent, paper (for example, filter paper) is impregnated with the resulting solution, and the solvent is removed by drying at room temperature to 60° C. to obtain a medium containing the terbium compound doped with other rare earth element. After drying, the solvent is removed by evaporation, but may remain. The use of a solvent is preferable because it makes it easier to impregnate the medium with the terbium compound doped with other rare earth element and makes it easier to adjust the concentration of the terbium compound doped with other rare earth element.

Examples of solvents that can be used to dissolve the terbium compound doped with other rare earth element, include, but are not limited to, dimethyl sulfoxide, methanol, ethanol, water, N,N-dimethylformamide, tetrahydrofuran, acetone, acetonitrile and 1,4-dioxane.

In order to effectively capture methyl salicylate, it is preferable to add the above-mentioned non-volatile ionic liquid to the solvent. When a non-volatile ionic liquid is used, after removing the solvent, the terbium compound doped with other rare earth element is dissolved in the non-volatile ionic liquid, but some of it may be precipitated. The terbium compound doped with other rare earth element can function as a receptor for methyl salicylate in the same way, whether it is dissolved in the non-volatile ionic liquid or precipitated.

The ratio of the ionic liquid to the solvent can be appropriately set for the medium to be impregnated. If the ratio of the ionic liquid to the solvent is too low, the amount of the ionic liquid in the medium after drying is reduced, and the effect of improving the detection sensitivity may be reduced. On the other hand, if the ratio of the ionic liquid to the solvent is too high, the ionic liquid may have a disadvantage of being difficult to impregnate into the medium due to its high viscosity. Therefore, the ratio of the ionic liquid to the solvent is properly set for the medium. For example, when impregnating filter paper, the ratio of the ionic liquid to the solvent is preferably 5 to 50% by weight, more preferably 10 to 30% by weight.

### (2) Detection Section

The detection section of the methyl salicylate sensor of the present disclosure is configured to optically detect the capture of methyl salicylate by the capture section. The detection section may be configured as a separate device rather than being an integral device with the capture section. In one aspect of the present disclosure, the optical detection section has an excitation light source (light emitter) and a detection element (fluorescence receiver) to detect the fluorescence emission of the complex formed by the terbium compound doped with other rare earth element and methyl salicylate, and can detect and/or measure the concentration of methyl salicylate based on the observed change in fluorescence intensity.

In one aspect of the present disclosure, the detection section may include a computer executing a program to process the detection and/or concentration measurement of methyl salicylate. Such a program may be, for example, a program that causes a computer to (i) receive a signal from an optical detecting element, (ii) determine the presence or absence and/or the concentration of methyl salicylate by analyzing the received signal, and (iii) output the analysis results.

In one aspect of the present disclosure, analysis of the received signal may include determining the presence or absence and/or the concentration of methyl salicylate, for example by comparing the received signal to a predetermined reference value. Also, in one aspect of the present disclosure, analysis results can be output to, for example, a display device connected to the sensor, or other equipment connected via a network.

In one aspect of the present disclosure, the methyl salicylate sensor of the present disclosure senses methyl salicylate, which is a plant hormone released when agricultural crops are infected with pathogens. Therefore, the methyl salicylate sensor of the present disclosure can be used as a sensor for detecting pathogen infection in plants including agricultural crops.

### [3] Method for Sensing Methyl Salicylate

One aspect of the present disclosure relates to a method for sensing methyl salicylate using fluorescence emission from a complex obtained by reacting a terbium compound doped with other rare earth element with methyl salicylate using the reagent or methyl salicylate sensor, wherein the method comprises:
(i) reacting a terbium compound doped with other rare earth element with methyl salicylate to form a complex;
(ii) exposing the complex to excitation light; and
(iii) detecting fluorescence emitted by the complex.

While the terbium compound doped with other rare earth element alone hardly exhibits fluorescence emission, the complex produced by the reaction between the terbium compound doped with other rare earth element and methyl salicylate newly exhibits fluorescence emission. Thus, methyl salicylate can be detected by utilizing this phenomenon.

In one aspect of the present disclosure, a suitable wavelength in the range of 300 to 400 nm is selected as the excitation wavelength. Further, in one aspect of the present disclosure, the determination of the concentration of methyl salicylate can also be performed by comparing the intensity of the detected fluorescence with a predetermined reference value.

### [4] Method for Detecting Pathogen Infection in Plant

One aspect of the present disclosure relates to a method for detecting pathogen infection in plants by placing the reagent or methyl salicylate sensor in the vicinity of a plant and confirming fluorescence emission derived from the complex formed by reaction between a terbium compound doped with other rare earth element and methyl salicylate.

Examples of the plant that may be the monitoring target include, but are not limited to, cucumber, watermelon, tomato, eggplant, green pepper, paprika, shishito pepper, melon, Chinese cabbage, cabbage, radish, lettuce, green onion, broccoli, onion, garlic, yam, asparagus, carrot, potato, celery, tobacco, rice, and strawberry.

Examples of the disease that may be detected include, but are not limited to, ring spot disease, leaf spot, Corynespora target spot, leaf mold, fusarium wilt, root rot wilt, Verticillium wilt, brown root rot, gray phytophthora rot, root rot, black dot root rot, southern blight, damping off, brown leaf spot, downy mildew, powdery mildew, gray mold, anthracnose, scab, Sclerotinia rot, gummy stem blight, leaf spot, blight, mosaic disease, spotted wilt, yellow leaf curl, bacterial wilt, bacterial soft rot, bacterial canker, pith necrosis, bacterial black spot, and bacterial leaf spot, and examples of the pathogen infection that may be detected include, but are not limited to, infections caused by the causative microorganisms of the above diseases.

In the context of the present disclosure, when referring to placing in the vicinity of a plant, examples of the term “vicinity” include, but are not limited to, a distance within 2 m, within 1 m, within 75 cm, within 50 cm, within 40 cm, within 30 cm, within 20 cm, within 10 cm, or within 5 cm of the plant to be monitored, and an appropriate distance is selected as appropriate in consideration of a variety of factors. A person skilled in the art would be able to set the position of the sensor to be installed as appropriate in consideration of a variety of conditions.

Further, one embodiment of the present disclosure relates to the use of a reagent or a methyl salicylate sensor in detection of pathogen infection in a plant. Moreover, one embodiment of the present disclosure relates to use of a terbium compound doped with other rare earth element in production of a reagent or a methyl salicylate sensor.

### EXAMPLES

The present disclosure will be described further in detail by way of examples below, but the present disclosure should not be limited by the following examples.

### Synthesis Example 1

Complex salt of terbium acetate (Tb(CH₃COO)₃:TbA) and terbium pivalate (Tb(t-C₄H₉COO)₃:TbPv) doped with 2% cerium (Ce):

[Ce_{0.02}Tb(CH₃COO)_{0.81}(C₄H₉COO)_{2.25}:0.02CeA-0.25TbA-0.75TbPv]

0.2 g of terbium acetate tetrahydrate, 0.6797 g of terbium pivalate, and 0.0131 g of cerium acetate monohydrate were dissolved in 120 ml of methanol, and heated under reflux for 5 hours. After cooling, the methanol was distilled off, and the precipitated white crystals were dried in a vacuum to obtain 0.733 g of the desired terbium compound doped with 2% cerium.

### Synthesis Example 2

Complex salt of terbium acetate (TbA) and terbium pivalate (TbPv) doped with 2% europium (Eu):

[Eu_{0.02}Tb(CH₃COO)_{0.81}(C₄H₉COO)_{2.25}:0.02EuA-0.25TbA-0.75TbPv]

The synthesis was carried out in the same manner as in Synthesis Example 1, except that 0.0131 g of cerium acetate monohydrate was replaced with 0.01573 g of europium acetate (CeA) tetrahydrate.

### Synthesis Example 3

Complex salt of terbium acetate (TbA) and terbium pivalate (TbPv) doped with 2% gadolinium (Gd):

[Gd_{0.02}Tb(CH₃COO)_{0.81}(C₄H₉COO)_{2.25}0.02GdA-0.25TbA-0.75TbPv]

The synthesis was carried out in the same manner as in Synthesis Example 1, except that 0.0131 g of cerium acetate monohydrate was replaced with 0.01593 g of gadolinium acetate (DyA) tetrahydrate.

### Synthesis Example 4

Complex salt of terbium acetate (TbA) and terbium pivalate (TbPv) doped with 2% dysprosium (Dy):

[Dy_{0.02}Tb(CH₃COO)_{0.81}(C₄H₉COO)_{2.25}:0.02DyA-0.25TbA-0.75TbPv]

The synthesis was carried out in the same manner as in Synthesis Example 1, except that 0.0131 g of cerium acetate monohydrate was replaced with 0.01614 g of dysprosium acetate (DyA) tetrahydrate.

### Synthesis Example 5

Complex salt of terbium acetate (TbA) and terbium pivalate (TbPv) doped with 2% ytterbium (Yb):

[Dy_{0.02}Tb(CH₃COO)_{0.81}(C₄H₉COO)_{2.25}:0.02DyA-0.25TbA-0.75TbPv]

The synthesis was carried out in the same manner as in Synthesis Example 1, except that 0.0131 g of cerium acetate monohydrate was replaced with 0.01655 g of ytterbium acetate (YbA) tetrahydrate.

### Synthesis Example 6

Complex salt of terbium acetate (TbA) and terbium pivalate (TbPv) doped with 1% Cerium (Ce):

[Ce_{0.01}Tb(CH₃COO)_{0.78}(C₄H₉COO)_{2.25}:0.01CeA-0.25TbA-0.75TbPv]

The synthesis was carried out in the same manner as in Synthesis Example 1, except that 0.0066 g of cerium acetate monohydrate was used.

### Synthesis Example 7

Complex salt of terbium acetate (TbA) and terbium pivalate (TbPv) doped with 1.5% cerium (Ce):

[Ce_{0.015}Tb(CH₃COO)_{0.795}(C₄H₉COO)_{2.25}:0.015CeA-0.25TbA-0.75TbPv]

The synthesis was carried out in the same manner as in Synthesis Example 1, except that 0.0098 g of cerium acetate monohydrate was used.

### Synthesis Example 8

Complex salt of terbium acetate (TbA) and terbium pivalate (TbPv) doped with 2.5% cerium (Ce):

[Ce_{0.025}Tb(CH₃COO)_{0.825}(C₄H₉COO)_{2.25}:0.025CeA-0.25TbA-0.75TbPv]

The synthesis was carried out in the same manner as in Synthesis Example 1, except that 0.0164 g of cerium acetate monohydrate was used.

### Synthesis Example 9

Complex salt of terbium acetate (TbA) and terbium pivalate (TbPv) doped with 4% cerium (Ce): [Ce_{0.04}Tb(CH₃COO)_{0.87}(C₄H₉COO)_{2.25}:0.04CeA-0.25TbA-0.75TbPv]

The synthesis was carried out in the same manner as in Synthesis Example 1, except that 0.0262 g of cerium acetate monohydrate was used.

### Synthesis Example 10

Complex salt of terbium acetate (TbA) and terbium pivalate (TbPv) doped with 5% cerium (Ce): [Ce_{0.05}Tb(CH₃COO)_{0.9}(C₄H₉COO)_{2.25}:0.05CeA-0.25TbA-0.75TbPv]

The synthesis was carried out in the same manner as in Synthesis Example 1, except that 0.0328 g of cerium acetate monohydrate was used.

### Synthesis Example 11

Complex salt of terbium acetate (TbA) and terbium pivalate (TbPv) doped with 6% cerium (Ce): [Ce_{0.06}Tb(CH₃COO)_{0.93}(C₄H₉COO)_{2.25}:0.06CeA-0.25TbA-0.75TbPv]

The synthesis was carried out in the same manner as in Synthesis Example 1, except that 0.0393 g of cerium acetate monohydrate was used.

### Synthesis Example 12

Complex salt of terbium acetate (TbA) and terbium pivalate (TbPv) doped with 2.5% ytterbium (Yb):

[Yb_{0.025}Tb(CH₃COO)_{0.825}(C₄H₉COO)_{2.25}:0.025YbA-0.25TbA-0.75TbPv]

The synthesis was carried out in the same manner as in Synthesis Example 5, except that 0.02069 g of ytterbium acetate (YbA) tetrahydrate was used.

### Synthesis Example 13

Complex salt of terbium acetate (TbA) and terbium pivalate (TbPv) doped with 4% ytterbium (Yb):

[Yb_{0.04}Tb(CH₃COO)_{0.87}(C₄H₉COO)_{2.25}:0.04YbA-0.25TbA-0.75TbPv]

The synthesis was carried out in the same manner as in Synthesis Example 5, except that 0.0331 g of ytterbium acetate (YbA) tetrahydrate was used.

### Synthesis Example 14

Terbium acetate (TbA) doped with 2% cerium (Ce):

[Ce_{0.02}Tb(CH₃COO)_{3.06}:0.02CeA-TbA]

1 g of terbium acetate tetrahydrate and 0.0164 g of cerium acetate monohydrate were dissolved in 100 ml of methanol, and heated under reflux for 5 hours. After cooling, the precipitated white crystals were dried in vacuum to obtain 0.411 g of the desired terbium acetate doped with 2% cerium.

### Synthesis Example 15

Terbium acetate (TbA) doped with 4% cerium (Ce):

[Ce_{0.04}Tb(CH₃COO)_{3.12}:0.04CeA-TbA]

The synthesis was carried out in the same manner as in Synthesis Example 14, except that 0.0328 g of cerium acetate monohydrate was used.

### Synthesis Example 16

Terbium nitrate (TbN) doped with 2% cerium (Ce):

[Ce_{0.02}Tb(NO₃)_{3.06}:0.02CeA-TbN]

1 g of terbium nitrate hexahydrate and 0.0192 g of cerium nitrate hexahydrate were dissolved in 50 ml of methanol, and heated under reflux for 5 hours. After cooling, the methanol was distilled off, and the solution was dried in vacuum at 60° C. to quantitatively obtain terbium nitrate doped with 2% cerium.

### Synthesis Example 17

Terbium nitrate (TbN) doped with 2% ytterbium (Yb):

[Yb_{0.02}Tb(NO₃)_{3.06}:0.02YbA-TbN]

1 g of terbium nitrate hexahydrate and 0.0198 g of ytterbium nitrate pentahydrate were dissolved in 50 ml of methanol, and heated under reflux for 5 hours. After cooling, the methanol was distilled off, and the solution was dried in vacuum at 60° C. to quantitatively obtain terbium nitrate doped with 2% ytterbium.

### Comparative Synthesis Example 1

Complex salt of terbium acetate (TbA) and terbium pivalate (TbPv) not doped with other rare earth element:

[Tb(CH₃COO)_{0.75}(C₄H₉COO)_{2.25}:0.25TbA-0.75TbPv]

0.2 g of terbium acetate tetrahydrate and 0.6797 g of terbium pivalate (the molar ratio of TbA to TbPv is 1:3) were heated under reflux for 5 hours in 65 ml of methanol. After cooling, the methanol was distilled off, and the precipitated white crystals were dried in a vacuum to obtain 0.74 g of the desired complex salt of terbium acetate and terbium pivalate.

### Example 1 and Comparative Example 1

[Fluorescence Emission Behavior Upon Reaction with Methyl Salicylate (MSA)]

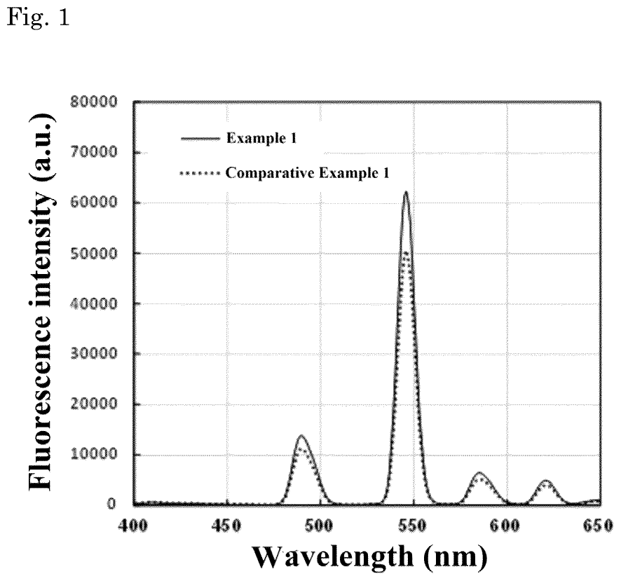

0.9 ml of DMSO solution of the complex salt of terbium acetate (TbA) and terbium pivalate (TbPv) doped with 2% cerium (0.02CeA-0.25TbA-0.75TbPv) obtained in Synthesis example 1 (the concentration 1.73 mM) and 0.1 ml of DMSO solution of methyl salicylate (MSA) (the concentration 1.5 mM) were mixed, and after 10 minutes, the mixture was diluted 20 times, 3 ml of the solution was placed in a quartz cell, and the fluorescence spectrum was measured at an excitation wavelength of 365 nm (Example 1). 0.9 ml of DMSO solution of 0.25TbA-0.75TbPv obtained in Comparative synthesis example 1 (the concentration 1.73 mM) and 0.1 ml of DMSO were mixed, and the mixture was diluted 20 times, 3 ml of the solution was placed in a quartz cell, and the fluorescence spectrum was measured at an excitation wavelength of 365 nm (Comparative Example 1). The obtained fluorescence spectrum curves are shown in FIG. 1. The solid line represents the fluorescence spectrum of 0.02CeA-0.25TbA-0.75TbPv+MSA (Example 1), and the dashed line represents the fluorescence spectrum of 0.25TbA-0.75TbPv (Comparative Example 1). It was found that the fluorescence intensity at a wavelength of 546 nm was increased by 23% by doping with cerium.

### Examples 2 to 5

Fluorescence spectra were measured in the same manner as in Example 1, except that the terbium compound obtained in Synthesis Example 1 was replaced with the terbium compounds obtained in Synthesis Examples 2 to 5. The fluorescence intensities at a wavelength of 546 nm in the obtained fluorescence spectra are summarized in Table 1. These results show that the fluorescence intensity increases when the terbium compound is doped with other rare earth element.

TABLE 1

| | | Fluorescence intensity at |
|---|---|---|
| | | wavelength 546 nm |
| | Terbium compound | (relative intensity) |
| Comparative | Comparative synthesis example 1: | 50580 (1) |
| Example 1 | 0.25TbA-0.75TbPv | |
| Examples 1 | Terbium compound obtained in | 62383 (1.23) |
| | Synthesis example 1: | |
| | 0.02CeA-0.25TbA-0.75TbPv | |
| Examples 2 | Terbium compound obtained in | 55497 (1.10) |
| | Synthesis example 2: | |
| | 0.02EuA-0.25TbA-0.75TbPv | |
| Examples 3 | Terbium compound obtained in | 55278 (1.09) |
| | Synthesis example 3: | |
| | 0.02GdA-0.25TbA-0.75TbPv | |
| Examples 4 | Terbium compound obtained in | 59772 (1.18) |
| | Synthesis example 4: | |
| | 0.02DyA-0.25TbA-0.75TbPv | |
| Examples 5 | Terbium compound obtained in | 61838 (1.22) |
| | Synthesis example 5: | |
| | 0.02YbA-0.25TbA-0.75TbPv | |

### Examples 6 to 9 and Comparative Examples 2 and 3

Fluorescence spectra were measured in the same manner as in Example 1, except that the terbium compound obtained in Synthesis Example 1 was replaced with the terbium compound obtained in Synthesis Examples 6 to 11. The fluorescence intensities at a wavelength of 546 nm in the obtained fluorescence spectra are summarized in Table 2. The fluorescence intensities in Examples 1 and 6 to 9, which was doped with other rare earth element (cerium), were higher than that of Comparative Example 1, which was not doped with other rare earth element (cerium). However, the fluorescence intensities in Comparative Example 2, in which the amount of cerium doped was too small, and in Comparative Example 3, in which the amount of cerium doped was too large, were lower than that of Comparative Example 1, which was not doped with cerium. From these results, it was found that a doping amount of other rare earth element of 1.5 to 5% (c in formula (1) is 0.015 to 0.05) is effective.

TABLE 2

| | | Fluorescence intensity |
|---|---|---|
| | | at wavelength 546 nm |
| | Cerium-doped terbium compound | (relative intensity) |
| Comparative | Comparative synthesis example 1: | 50580 (1) |
| Example 2 | 0.25TbA-0.75TbPv | |
| Comparative | Terbium compound obtained in | 30105 (0.60) |
| Example 3 | Synthesis example 6: | |
| | 0.01CeA-0.25TbA-0.75TbPv | |
| Examples 6 | Terbium compound obtained in | 57865 (1.14) |
| | Synthesis example 7: | |
| | 0.015CeA-0.25TbA-0.75TbPv | |
| Examples 1 | Terbium compound obtained in | 62383 (1.23) |
| | Synthesis example 1: | |
| | 0.02CeA-0.25TbA-0.75TbPv | |
| Examples 7 | Terbium compound obtained in | 64249 (1.27) |
| | Synthesis example 8: | |
| | 0.025CeA-0.25TbA-0.75TbPv | |
| Examples 8 | Terbium compound obtained in | 56144 (1.11) |
| | Synthesis example 9: | |
| | 0.04CeA-0.25TbA-0.75TbPv | |
| Examples 9 | Terbium compound obtained in | 53249 (1.05) |
| | Synthesis example 10: | |
| | 0.05CeA-0.25TbA-0.75TbPv | |
| Comparative | Terblum compound obtained in | 43948 (0.87) |
| Example 3 | Synthesis example 11: | |
| | 0.06CeA-0.25TbA-0.75TbPv | |

### Example 10

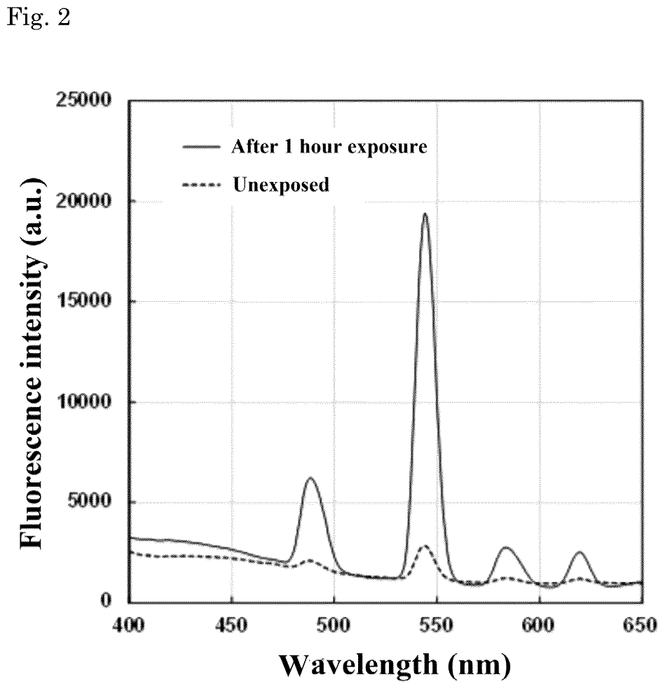

0.0437 g of terbium acetate-terbium pivalate complex salt doped with 2% cerium (0.02CeA-0.25TbA-0.75TbPv) obtained in Synthesis example 1 was dissolved in 1.6 ml of dimethyl sulfoxide (DMSO), and 0.4 ml of 1-etyl-1-methylimidazolium acetate (EMImAc) was added thereto (DMSO/EMImAc=8/2). 0.2 ml of the solution was dropped onto a circular filter paper (Φ40 mm) and dried to volatilize the DMSO, thereby obtaining a filter paper containing 0.02CeA-0.25TbA-0.75TbPv and EMImAc. The obtained filter paper was placed in a desiccator with a capacity of 800 ml, and methyl salicylate at a concentration of 5 ppb was introduced therein using a permeator PD-1B-2 (manufactured by Gastec Corporation) with nitrogen as a carrier gas, and after one hour of exposure, the filter paper was removed and the fluorescence spectrum was measured at an excitation wavelength of 365 nm. As shown in FIG. 2, the fluorescence intensity at a wavelength of 544 nm in the fluorescence spectrum after one hour of exposure was about 11 times that of the unexposed sample, demonstrating that gas-phase methyl salicylate at a concentration of 5 ppb could be sensed.

### Example 11

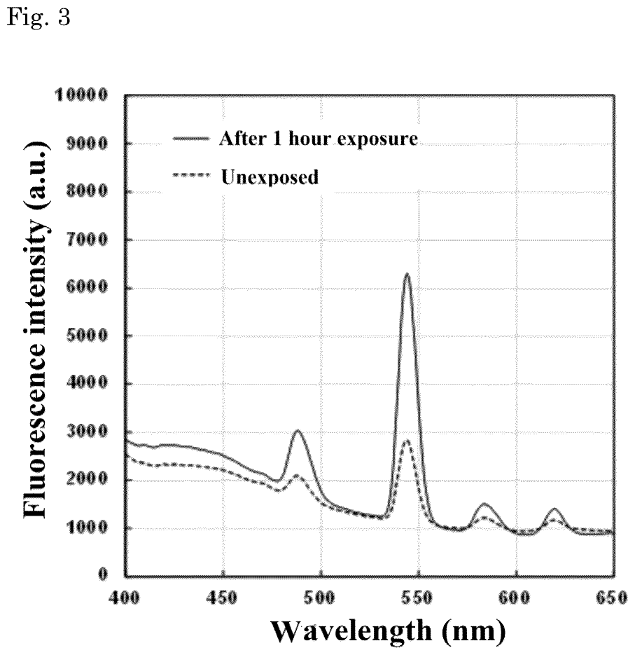

Evaluation was performed in the same manner as in Example 10, except that the terbium acetate-terbium pivalate complex salt doped with 2% of gadolinium (0.02GdA-0.25TbA-0.75TbPv) obtained in Synthesis example 3 was used instead of the terbium acetate-terbium pivalate complex salt doped with 2% cerium (0.02CeA-0.25TbA-0.75TbPv) obtained in Synthesis example 1. As shown in FIG. 3, the fluorescence intensity at a wavelength of 544 nm in the fluorescence spectrum after one hour of exposure was about 3 times that of the unexposed sample, demonstrating that gas-phase methyl salicylate at a concentration of 5 ppb could be sensed.

### Example 12

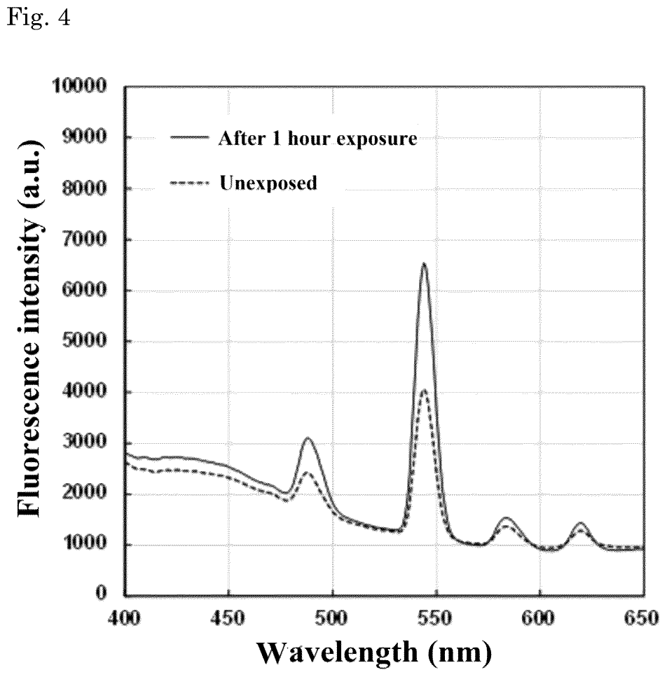

0.0438 g of terbium acetate-terbium pivalate complex salt doped with 2% ytterbium (0.02YbA-0.25TbA-0.75TbPv) obtained in Synthesis example 5 was dissolved in 1.6 ml of dimethyl sulfoxide (DMSO), and 0.4 ml of 1-etyl-1-methylimidazolium acetate (EMImAc) was added thereto (DMSO/EMImAc=8/2). 0.2 ml of the solution was dropped onto a circular filter paper (Φ40 mm) and dried to volatilize the DMSO, thereby obtaining a filter paper containing 0.02YbA-0.25TbA-0.75TbPv and EMImAc. The obtained filter paper was placed in a desiccator with a capacity of 800 ml, and methyl salicylate at a concentration of 2 ppb was introduced therein using a permeator PD-1B-2 with nitrogen as a carrier gas, and after one hour of exposure, the filter paper was removed and the fluorescence spectrum was measured at an excitation wavelength of 365 nm. As shown in FIG. 4, the fluorescence intensity at a wavelength of 544 nm in the fluorescence spectrum after one hour of exposure was about 2 times that of the unexposed sample, demonstrating that gas-phase methyl salicylate at a concentration of 2 ppb could be sensed.

### Example 13 and Comparative Example 4

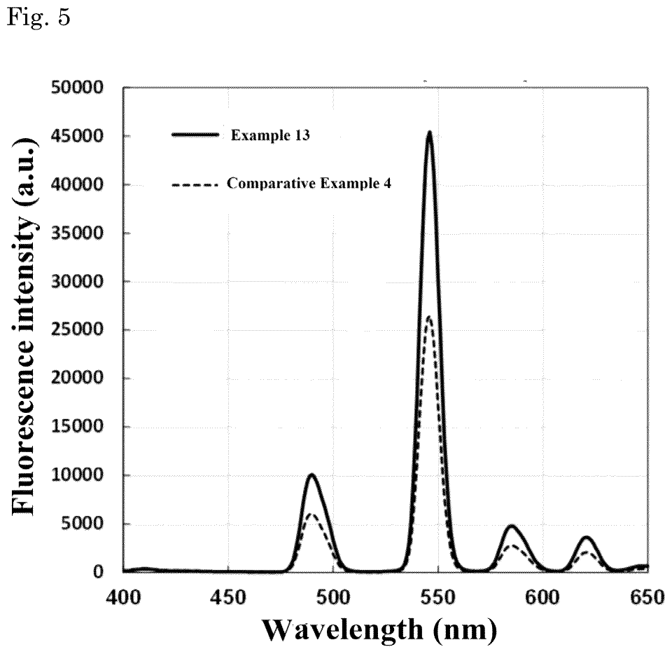

The fluorescence spectrum was measured in the same manner as in Example 1, except that the terbium compound obtained in Synthesis Example 1 was replaced with the terbium compound obtained in Synthesis Example 14. As Comparative Example 4, the fluorescence spectrum was similarly measured using terbium acetate tetrahydrate. The obtained fluorescence spectrum curves are shown in FIG. 5. The solid line represents the fluorescence spectrum of the terbium compound in Synthesis Example 14+MSA (Example 13), and the dashed line represents the fluorescence spectrum of terbium acetate+MSA (Comparative Example 4). In the obtained fluorescence spectrum, it was found that the fluorescence intensity at a wavelength of 546 nm was increased by 72% by doping with cerium.

### Example 14

The fluorescence spectrum was measured in the same manner as in Example 13, except that the terbium compound obtained in Synthesis Example 14 was replaced with the terbium compound obtained in Synthesis Example 15. In the obtained fluorescence spectrum, it was found that the fluorescence intensity at a wavelength of 546 nm was increased by 88% by doping with 4% cerium.

### Examples 15 and 16 and Comparative Example 5

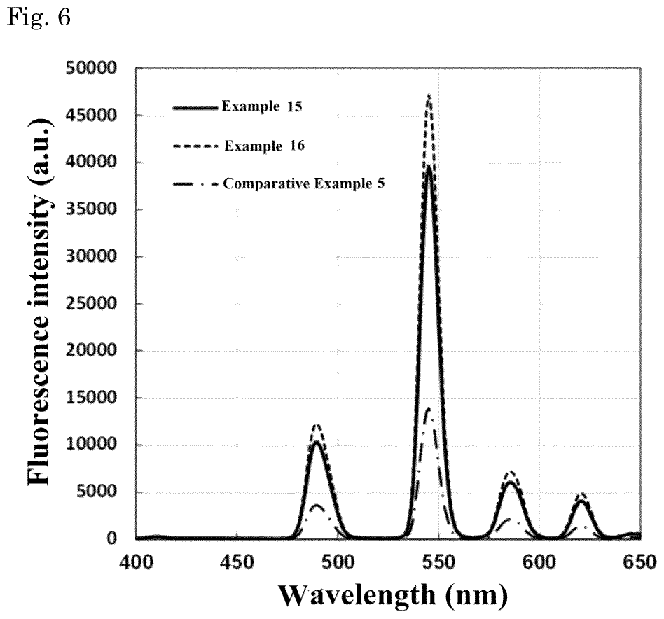

The fluorescence spectrum was measured in the same manner as in Example 1, except that the terbium compound obtained in Synthesis Example 1 was replaced with the terbium compound obtained in Synthesis Example 16. Furthermore, the fluorescence spectrum was measured in the same manner for the terbium compound obtained in Synthesis Example 17. In addition, as Comparative Example 5, the fluorescence spectrum was measured in the same manner using terbium nitrate hexahydrate. The obtained fluorescence spectrum curves are shown in FIG. 6. The solid line represents the fluorescence spectrum of the terbium compound of Synthesis Example 16+MSA (Example 15), the dashed line represents the fluorescence spectrum of the terbium compound of Synthesis Example 17+MSA (Example 16), and the dashed-dotted line represents the fluorescence spectrum of terbium nitrate+MSA (Comparative Example 5). In the obtained fluorescence spectrum, it was found that the fluorescence intensity at a wavelength of 545 nm increased 2.9 times by doping with 2% cerium, and increased 3.4 times by doping with 2% ytterbium.

Although the present disclosure has been described with reference to the embodiments and Examples, the present disclosure is not limited to the above embodiments and Examples. Various modifications can be made to the configuration and details of the present disclosure within the scope of the present disclosure that can be understood by those skilled in the art.

### Supplementary Note 1

A reagent for detecting methyl salicylate, comprising a terbium compound doped with rare earth element other than terbium.

### Supplementary Note 2

The reagent according to supplementary note 1, wherein the terbium compound doped with rare earth element other than terbium is represented by the following general formula (1):

Ln_{c}TbXₐY_{b} (1)

(wherein Ln represents at least one rare earth element other than terbium, X and Y each independently represent at least one selected from the group consisting of acetate ion, propionate ion, butyrate ion, pivalate ion, 2-methylbutyrate ion, trifluoroacetate ion, benzoate ion, chloride ion, and nitrate ion, c represents a number ranging from 0.015 to 0.05, and a and b each independently represent a number that satisfies 0≤a≤3.15, 0≤b≤3.15, and a+b=3+3×c.)

### Supplementary Note 3

The reagent according to supplementary note 1 or 2, wherein the rare earth element other than terbium is at least one selected from the group consisting of scandium, yttrium, lanthanum, cerium, praseodymium, neodymium, promethium, samarium, europium, gadolinium, dysprosium, holmium, erbium, thulium, ytterbium, and lutetium.

### Supplementary Note 4

The reagent according to any of preceding supplementary notes, further comprising a non-volatile ionic liquid.

### Supplementary Note 5

The reagent according to supplementary note 4, wherein the non-volatile ionic liquid is at least one selected from the group consisting of 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium methyl dicyanamate.

### Supplementary Note 6

A methyl salicylate sensor for detecting methyl salicylate, comprising:
a capture section of methyl salicylate having a reagent comprising a terbium compound doped with rare earth element other than terbium, and
a detection section detecting that methyl salicylate has been captured by the capture section.

### Supplementary Note 7

The methyl salicylate sensor according to supplementary note 6, wherein the capture section comprises a medium containing the reagent.

### Supplementary Note 8

The methyl salicylate sensor according to supplementary note 7, wherein the medium is paper, glass fiber, resin, or water-soluble polymer.

### Supplementary Note 9

A method for sensing methyl salicylate, comprising detecting methyl salicylate using the reagent according to any of supplementary note 1 to 5, comprising:
(i) reacting a terbium compound doped with rare earth element other than terbium with methyl salicylate to form a complex;
(ii) exposing the complex to excitation light; and
(iii) detecting fluorescence emitted by the complex.

### Supplementary Note 10

A method for detecting pathogen infection in a plant by placing the reagent according to any of supplementary note 1 to 5, in the vicinity of a plant and confirming fluorescence emission derived from a complex formed by a reaction between a terbium compound doped with a rare earth element other than terbium and methyl salicylate.

### Supplementary Note 11

The method according to supplementary note 10, wherein the vicinity of a plant is a distance within 2 m of the plant to be monitored.

### INDUSTRIAL APPLICABILITY

The methyl salicylate sensor for detecting methyl salicylate, which is a plant hormone of the present disclosure, can selectively detect methyl salicylate, which is a plant hormone released by plants during pathogen infection, because it efficiently captures methyl salicylate, forms a complex, and emits fluorescence by having a capture section comprising a medium comprising a terbium compound doped with other rare earth element. In addition, pathogen infection in a plant can be detected by using the methyl salicylate sensor. Specifically, the methyl salicylate sensor can be used as a new sensor for agricultural ICT in facility horticulture such as greenhouses, which can detect pathogen infection of agricultural crops.

1. A reagent for detecting methyl salicylate, comprising a terbium compound doped with rare earth element other than terbium.

2. The reagent according to claim 1, wherein the terbium compound doped with rare earth element other than terbium is represented by the following general formula (1):
Ln_{c}TbXₐY_{b} (1)
(wherein Ln represents at least one rare earth element other than terbium, X and Y each independently represent at least one selected from the group consisting of acetate ion, propionate ion, butyrate ion, pivalate ion, 2-methylbutyrate ion, trifluoroacetate ion, benzoate ion, chloride ion, and nitrate ion, c represents a number ranging from 0.015 to 0.05, and a and b each independently represent a number that satisfies 0≤a≤3.15, 0≤b≤3.15, and a+b=3+3×c).

3. The reagent according to claim 1, wherein the rare earth element other than terbium is at least one selected from the group consisting of scandium, yttrium, lanthanum, cerium, praseodymium, neodymium, promethium, samarium, europium, gadolinium, dysprosium, holmium, erbium, thulium, ytterbium, and lutetium.

4. The reagent according to claim 1, further comprising a non-volatile ionic liquid.

5. The reagent according to claim 4, wherein the non-volatile ionic liquid is at least one selected from the group consisting of 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium methyl dicyanamate.

6. A methyl salicylate sensor for detecting methyl salicylate, comprising:
a capture section of methyl salicylate having a reagent comprising a terbium compound doped with rare earth element other than terbium, and
a detection section detecting that methyl salicylate has been captured by the capture section.

7. The methyl salicylate sensor according to claim 6, wherein the capture section comprises a medium containing the reagent.

8. The methyl salicylate sensor according to claim 7, wherein the medium is paper, glass fiber, resin, or water-soluble polymer.

9. A method for sensing methyl salicylate, comprising detecting methyl salicylate using the reagent according to claim 1, comprising:
(i) reacting a terbium compound doped with rare earth element other than terbium with methyl salicylate to form a complex;
(ii) exposing the complex to excitation light; and
(iii) detecting fluorescence emitted by the complex.

10. A method for detecting pathogen infection in a plant by placing the reagent according to claim 1 in the vicinity of a plant and confirming fluorescence emission derived from a complex formed by a reaction between a terbium compound doped with a rare earth element other than terbium and methyl salicylate.

11. The method according to claim 10, wherein the vicinity of a plant is a distance within 2 m of the plant to be monitored.

12. The reagent according to claim 2, wherein the rare earth element other than terbium is at least one selected from the group consisting of scandium, yttrium, lanthanum, cerium, praseodymium, neodymium, promethium, samarium, europium, gadolinium, dysprosium, holmium, erbium, thulium, ytterbium, and lutetium.

13. The reagent according to claim 2, further comprising a non-volatile ionic liquid.

14. A method for sensing methyl salicylate, comprising detecting methyl salicylate using the reagent according to claim 2, comprising:
(i) reacting a terbium compound doped with rare earth element other than terbium with methyl salicylate to form a complex;
(ii) exposing the complex to excitation light; and
(iii) detecting fluorescence emitted by the complex.

15. A method for detecting pathogen infection in a plant by placing the reagent according to claim 2 in the vicinity of a plant and confirming fluorescence emission derived from a complex formed by a reaction between a terbium compound doped with a rare earth element other than terbium and methyl salicylate.

16. The method according to claim 15, wherein the vicinity of a plant is a distance within 2 m of the plant to be monitored.
